(19) United States
(12) Patent Application Publication
(10) Pub. No.: US 2006/0251638 A1
(43) Pub. Date: Nov. 9, 2006

(54) Cytoprotection through the use of hif hydroxylase inhibitors

(76) Inventors: Volkmar Guenzler-Pukall, San Leandro, CA (US); Stephen J. Klaus, San Francisco, CA (US); David Y. Liu, Palo Alto, CA (US); Todd W. Seeley, Moraga, CA (US)

Correspondence Address: FIBROGEN, INC.;INTELLECTUAL PROPERTY DEPARTMENT, 225 GATEWAY BOULEVARD, SOUTH SAN FRANCISCO, CA, 94080, US

(21) Appl. No.: 10/554,450

(22) PCT Filed: Jun. 4, 2004
(86) PCT No.: PCT/US04/17689

§ 371 Date: Oct. 25, 2005

Related U.S. Application Data
(60) Provisional application No. 60/476,740, filed on Jun. 6, 2003.
(60) Provisional application No. 60/476,723, filed on Jun. 6, 2003.
(60) Provisional application No. 60/554,568, filed on Mar. 19, 2004.

(51) Int. Cl. A61K 38/44; A61K 38/22

(52) U.S. Cl. 424/94.4; 514/2

(57) ABSTRACT

The invention relates to methods for conferring cytoprotection, or for inducing a cytoprotective effect, by administering a compound that inhibits HIF hydroxylase. Compounds for use in these methods are also provided.

This application claims the benefit of U.S. Provisional Application Ser. No. 60/476,740, filed on 6 Jun. 2003; U.S. Provisional Application Ser. No. 60/476,723, filed on 6 Jun. 2003; and U.S. Provisional Application Ser. No. 60/554,568, filed on 19 Mar. 2004, each of which is incorporated by reference herein in its entirety.

### FIELD OF THE INVENTION

The invention relates to methods for conferring cytoprotection, or for inducing a cytoprotective effect, by administering a compound that inhibits HIF hydroxylase. Compounds for use in these methods are also provided.

### BACKGROUND

Cytoprotection refers to the ability of natural and/or therapeutic agents to protect a cell against damage and death. Cells have developed certain adaptive mechanisms, triggered in response to stress, that extend viability, delaying or preventing apoptosis or cell death. In many instances, however, natural cytoprotective mechanisms are insufficient, inadequate, or induced too late to provide necessary benefit, e.g., cell survival, reduced tissue and organ damage, etc. As a result, cell death may occur by apoptotic or necrotic mechanisms.

Cell damage and cell death can result from stress conferred by various physiological and environmental factors. These factors can include, for example, exposure to radiation (UV, gamma), cellular toxins and waste products, environmental toxins, free radicals, and reactive oxygen species; hypoxia or oxygen deprivation; nutrient deprivation; growth factor withdrawal, etc. Certain medical events and procedures, e.g., surgical trauma, including transplantation events, etc., or various therapies, including radiation therapy and chemotherapy, can involve exposure of cells to various stresses and/or cytotoxic agents. Physiological conditions including infection, inflammation, malignancies, and other diseases, or events such as ischemic events, or traumatic injury, can compromise function and viability.

Progressive damage to cells and consequently tissues and organs is a common feature of degenerative disorders and diseases, trauma, and the process of aging in animals. Alterations in cell survival contribute to the pathogenesis of numerous conditions and disorders, including infections, inflammation, malignancies, and other conditions; e.g., cancer, viral and bacterial infections, autoimmune diseases, immunodeficiency disorders (e.g., AIDS, etc.), aging and associated disorders, neurodegenerative disorders (Alzheimer's disease, Parkinson's disease, amyotrophic laterial sclerosis, retinitis pigmentosa, cerebellar degeneration), myelodysplastic syndromes (aplastic anemia), heart disease, cardiac injury including ischemic injury (myocardial infarction, stroke, reperfusion injury), toxin-induced liver disease, etc.

The ability to induce and/or enhance innate cytoprotective mechanisms, to precondition against future trauma (e.g., surgery, etc.), as part of a combinatorial therapy (e.g., to counteract some cytotoxic aspects of an agent administered in chemotherapy, etc.), and to ameliorate the consequences of exposure to physiological and/or environmental stresses, would be beneficial.

The present invention answers this need by providing methods for conferring cytoprotection on and for inducing or enhancing cytoprotective effects. In particular, the invention provides methods and compositions for the protection of cells, tissues, organs, and organisms, in vivo and in vitro.

### SUMMARY OF THE INVENTION

The invention provides a method for conferring cytoprotection on a cell, the method comprising administering to the cell an effective amount of a compound that inhibits HIF hydroxylase activity. A method for inducing a cytoprotective effect in a cell, the method comprising administering to the cell an effective amount of a compound that inhibits HIF hydroxylase activity, is also provided. In various embodiments, the cytoprotective effect is selected from the group consisting of increased energy preservation, increased anaerobic respiration, reduced oxygen consumption, reduced oxidative damage, prevention or reduction of apoptosis and inhibition of pro-apoptotic activities, and increased expression of cytoprotective factors, such as EPO and VEGF. In particular, the present invention provides methods and compounds for use in inducing HIF-regulated factors associated with cytoprotective processes including, e.g., angiogenic factors, modulators of apoptosis, regulators of energy consumption, anti-oxidant factors, and other cyto- and tissue-protective agents, etc.

The compounds of the invention are compounds that inhibit HIF hydroxylase activity. In one embodiment, the compound of the invention is selected from the group consisting of phenanthrolines; heterocyclic carbonyl glycines including, but not limited to, substituted quinoline-2-carboxamides and isoquinoline-3-carboxamides; and N-substituted arylsulfonylamino hydroxamic acids. In preferred embodiments, the compound of the invention is selected from the group consisting of 4-Oxo-1,4-dihydro-[1,10]phenanthroline-3-carboxylic acid (Compound A), 3-{[4-(3,3-Dibenzyl-ureido)-benzenesulfonyl]-[2-(4-methoxy-phenyl)-ethyl]-amino}-N-hydroxy-propionamide (Compound B), [(4-Hydroxy-7-phenylsulfanyl-isoquinoline-3-carbonyl)-amino]-acetic acid (Compound C), [(4-Hydroxy-6-phenylsulfanyl-isoquinoline-3-carbonyl)-amino]-acetic acid (Compound D), [(1-Chloro-4-hydroxy-7-phenoxy-isoquinoline-3-carbonyl)-amino]-acetic acid (Compound E), [(1-Bromo-4-hydroxy-7-phenoxy-isoquinoline-3-carbonyl)-amino]-acetic acid (Compound F), [(4-Hydroxy-7-phenoxy-isoquinoline-3-carbonyl)-amino]-acetic acid (Compound G), and [(1-Chloro-4-hydroxy-isoquinoline-3-carbonyl)-amino]-acetic acid (Compound H).

The invention provides a method for reducing or preventing apoptosis in a subject, the method comprising administering to the subject an effective amount of a compound that inhibits HIF hydroxylase activity. In one aspect, the reduction or prevention of apoptosis comprises inducing expression of anti-apoptotic factors. In various aspects, the anti-apoptotic factor is selected from the group consisting of adrenomedullin, heme oxygenase-1, and HSP70. Increases in expression of these anti-apoptotic factors can be measured by any of the methods available to one of skill in the art, including, e.g., measuring gene expression using microarray analysis, or by measuring protein expression using ELISA or other immunoassays, etc. The reduction or prevention of apoptosis can be measured by, e.g., reduced annexin V immunostaining of the cell. In specific aspects, the compound is selected from the group of compounds consisting of Compound C and Compound D.

In another aspect, the reduction or prevention of apoptosis comprises decreasing expression of pro-apoptotic factors. In one aspect, the pro-apoptotic factor is selected from the group consisting of caspase-3 and caspase-7. Decreased expression of pro-apoptotic factors can be measured by any of the methods available to one of skill in the art, including, e.g., using commercially available assays or kits, such as a commercially available fluorometric assay, etc. The reduction or prevention of apoptosis can be measured by, e.g., reduced annexin V immunostaining of the cell. In one preferred aspect, the compound is Compound G.

In one aspect, a method for reducing or preventing oxidative damage in a subject, the method comprising administering to the subject an effective amount of a compound that inhibits HIF hydroxylase activity is provided. In one aspect, the reduction or prevention of oxidative damage comprises inducing expression of factors having anti-oxidant activity. In various aspects, the factor having anti-oxidant activity is selected from the group consisting of adrenomedullin, heme oxygenase-1, and HSP70. Increases in expression of these factors can be measured by any of the methods available to one of skill in the art, including, e.g., measuring gene expression using microarray analysis, or by measuring protein expression using ELISA or other immunoassays, etc. The reduction or prevention of oxidative damage can be measured by, e.g., increased cell viability, for example, in a standard model of oxidative stress. In specific aspects, the compound is selected from the group of compounds consisting of Compound C and Compound D.

The invention provides a method for increasing energy preservation in a subject, the method comprising administering to the subject an effective amount of a compound that inhibits HIF hydroxylase activity. Methods for increasing energy preservation in a subject, wherein the subject has low glucose levels, or wherein the subject has impaired oxidative respiration, are specifically contemplated. In one embodiment, the energy preservation is ATP preservation. ATP preservation can be measured, e.g., by any the methods available in the art, such as by using standard available commercial kits, etc. In certain embodiments, the compound is selected from the group consisting of Compound A, Compound B, Compound C, Compound D, Compound E, Compound F, Compound G, and Compound H.

In any of the above methods, the compounds of the invention are compounds that inhibit HIF hydroxylase activity. In one embodiment, the compound of the invention is selected from the group consisting of phenanthrolines; heterocyclic carbonyl glycines including, but not limited to, substituted quinoline-2-carboxamides and isoquinoline-3-carboxamides; and N-substituted arylsulfonylamino hydroxamic acids. In preferred embodiments, the compound of the invention is selected from the group consisting of 4-Oxo-1,4-dihydro-[1,10]phenanthroline-3-carboxylic acid (Compound A), 3-{[4-(3,3-Dibenzyl-ureido)-benzenesulfonyl]-[2-(4-methoxy-phenyl)-ethyl]-amino}-N-hydroxy-propionamide (Compound B), [(4-Hydroxy-7-phenylsulfanyl-isoquinoline-3-carbonyl)-amino]-acetic acid (Compound C), [(4-Hydroxy-6-phenylsulfanyl-isoquinoline-3-carbonyl)-amino]-acetic acid (Compound D), [(1-Chloro-4-hydroxy-7-phenoxy-isoquinoline-3-carbonyl)-amino]-acetic acid (Compound E), [(1-Bromo-4-hydroxy-7-phenoxy-isoquinoline-3-carbonyl)-amino]-acetic acid (Compound F), [(4-Hydroxy-7-phenoxy-isoquinoline-3-carbonyl)-amino]-acetic acid (Compound G), and [(1-Chloro-4-hydroxy-isoquinoline-3-carbonyl)-amino]-acetic acid (Compound H).

In various embodiments, the present invention provides formulations or medicaments or pharmaceutical compositions comprising the compounds of the invention, and methods for the manufacture and use of such formulations or medicaments or pharmaceutical compositions. In one embodiment, a pharmaceutical composition is provided, wherein the pharmaceutical composition comprises a compound that inhibits HIF hydroxylase activity. In another embodiment, the invention encompasses a kit that comprises at least one compound that inhibits HIF hydroxylase activity.

### BRIEF DESCRIPTION OF THE DRAWINGS

FIG. 1 sets forth data showing decreased caspase activity in cells treated with a compound of the present invention.

FIG. 2 sets forth data showing preservation of ATP levels in cells treated with a compound of the present invention.

FIG. 3 sets forth data showing increased viability of cells treated with a compound of the present invention.

FIG. 4 sets forth data showing methods and compounds of the present invention decreased apoptosis in cells.

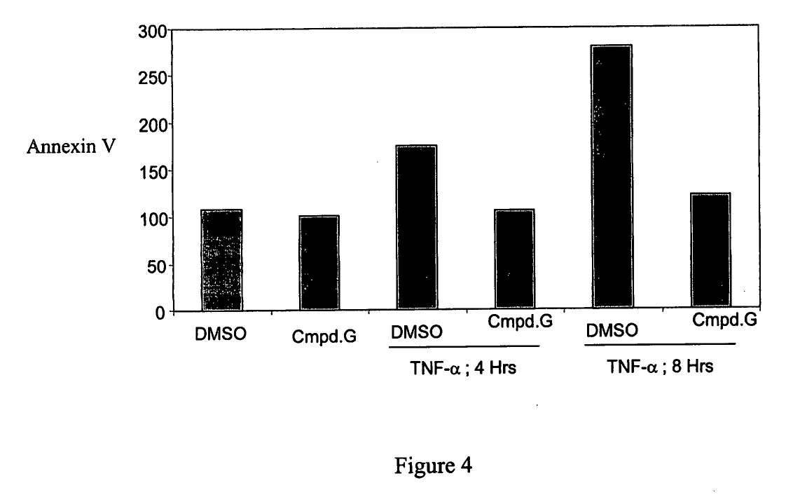

FIG. 5 sets forth data showing increased viability of cells treated with a compound of the present invention.

FIG. 6 sets forth data showing methods and compounds of the present invention increased heme oxygenase-1 expression.

### DETAILED DESCRIPTION

The present invention relates to methods and compounds for inducing a cytoprotective effect in a subject. The subject can be, e.g., a cell, a population of cells, a tissue, an organ, or an organism. The cytoprotective effect can be induced, as appropriate, in vivo or in vitro. It is explicitly contemplated that cytoprotection might desirably be induced under situations in which HIF-regulated cytoprotective effects would not be induced through natural mechanisms, including conditions of normal or adequate oxygen.

The present methods and compounds provide cytoprotection to cells, tissues, and organs by inducing in coordinate fashion specific cytoprotective effects. Coordinated induction refers to the ability of the present methods and compounds to induce in a subject a series of cytoprotective effects, sequentially or in parallel, that contribute to the viability of the subject. These desirable cytoprotective effects include increased energy preservation, increased anaerobic respiration, reduced oxygen consumption, reduced oxidative damage, prevention or reduction of apoptosis and inhibition of pro-apoptotic activities, and increased expression of cytoprotective factors, such as EPO and VEGF. In particular, the present invention provides methods and compounds for use in inducing HIF-regulated factors associated with cytoprotective processes including, e.g., angiogenic factors, modulators of apoptosis, regulators of energy consumption, anti-oxidant factors, and other cyto- and tissue-protective agents, etc.

Hypoxia inducible factor (HIF) is a transcriptional activator that mediates changes in gene expression in response to changes in cellular oxygen concentration. HIF is a heterodimer containing an oxygen-regulated alpha subunit (HIFα) and a constitutively expressed beta subunit (HIFβ), also known as aryl hydrocarbon receptor nuclear transporter (ARNT). In oxygenated (normoxic) cells, HIFα subunits are rapidly degraded by a mechanism that involves ubiquitination by the von Hippel-Lindau tumor suppressor (pVHL) E3 ligase complex. Under hypoxic conditions, HIFα is not degraded, and an active HIFα/β complex is formed.

The term “HIFα” refers to the alpha subunit of hypoxia inducible factor protein or to a fragment thereof. HIFαmay be any human or other mammalian protein, or fragment thereof, including human HIF-1α (Genbank Accession No. Q16665), HIF-2α (Genbank Accession No. AAB41495), and HIF-3α (Genbank Accession No. AAD22668); murine HIF-1α (Genbank Accession No. Q61221), HIF-2α (Genbank Accession No. BAA20130 and AAB41496), and HIF-3α (Genbank Accession No. AAC72734); rat HIF-1α (Genbank Accession No. CAA70701), HIF-2α (Genbank Accession No. CAB96612), and HIF-3α (Genbank Accession No. CAB96611); and cow HIF-1α (Genbank Accession No. BAA78675). HIFα may also be any non-mammalian protein or fragment thereof, including Xenopus laevis HIF-1α (Genbank Accession No. CAB96628), Drosophila melanogaster HIF-1α (Genbank Accession No. JC4851), and chicken HIF-1α (Genbank Accession No. BAA34234). HIFα gene sequences may also be obtained by routine cloning techniques, for example by using all or part of a HIFα gene sequence described above as a probe to recover and determine the sequence of a HIFα gene in another species.

Fragments of HIFα include the regions defined by human HIF-1α from amino acid 401 to 603 (Huang et al. (1998) Proc Natl Acad Sci USA 95:7987-7992), amino acid 531 to 575 (Jiang et al. (1997) J Biol Chem 272:19253-19260), amino acid 556 to 575 (Tanimoto et al. (2000) EMBO J. 19:4298-4309), amino acid 557 to 571 (Srinivas et al. (1999) Biochem Biophys Res Commun 260:557-561), and amino acid 556 to 575 (Ivan and Kaelin (2001) Science 292:464-468). Further, a fragment of HIFα includes any fragment containing at least one occurrence of the motif LXXLAP, e.g., as occurs in the HIFα native sequence at L₃₉₇TLLAP and L₅₅₉EMLAP. Additionally, a fragment of HIFα includes any fragment retaining at least one functional or structural characteristic of HIFα.

“Amino acid sequence” or “polypeptide” as used herein, e.g., to refer to HIFα and fragments thereof, refer to an oligopeptide, peptide, or protein sequence, or to a fragment of any of these, and to naturally occurring or synthetic molecules. “Fragments” can refer to any portion of a sequence that retains at least one structural or functional characteristic of the protein. Immunogenic fragments or antigenic fragments refer to fragments of polypeptides, preferably, fragments of about five to fifteen amino acids in length, that retain at least one biological or immunological activity. Where “amino acid sequence” is recited to refer to the polypeptide sequence of a naturally occurring protein molecule, “amino acid sequence” and like terms are not meant to limit the amino acid sequence to the complete native sequence associated with the recited protein molecule.

The destabilization of HIFα in normoxic environments is due to hydroxylation of specific proline residues by HIF-specific proline hydroxylases (HIF PHs). HIF-regulated genes encompass a variety of factors involved in numerous processes, including angiogenesis, erythropoiesis, glucose metabolism, and numerous cytoprotective and tissue protective mechanisms involved in producing, e.g., anti-apoptotic and anti-oxidative effects, etc. These include, e.g., glycolytic enzymes, glucose transporter (GLUT)-1, erythropoietin (EPO), and vascular endothelial growth factor (VEGF). (Jiang et al. (1996) J Biol Chem 271:17771-17778; Iliopoulus et al. (1996) Proc Natl Acad Sci USA 93:10595-10599; Maxwell et al. (1999) Nature 399:271-275; Sutter et al. (2000) Proc Natl Acad Sci USA 97:4748-4753; Cockman et al. (2000) J Biol Chem 275:25733-25741; and Tanimoto et al. (2000) EMBO J 19:4298-4309.)

Due to its regulation of such factors, HIF has been associated with various cytoprotective events, including preventing or reducing apoptosis. Iron chelators, e.g., deferoxamine, at high concentrations have been shown to protect against apoptosis induced by oxidative stress and glutathione depletion in neuronal cells, presumably due to stabilization of HIF-1, although this effect is consistent with the known ability of chelators to diminish hydroxyl radical formation. (Zaman et al; J. Neurosci 1999 19(22):9821-9830.) Cobalt chloride, which appears to activate HIF in corticoid cultures although it is not known to be a HIF-PH inhibitor, also protected against oxidative stress-induced death in these cells. (Zaman, supra.)

Hypoxia-induced Akt activation protected against apoptosis in rat PC12 cells subjected to serum withdrawal and chemotherapy, an effect observed also by treatment with deferoxamine, a compound known to mimic some effects of hypoxia. (Alvarez-Tejado et al. (2001) J Biol Chem 276:22368-22374.)

It has further been noted that HIF also stimulates anti-apoptotic protective signaling pathways mediated by Jak kinases and STAT transcription factors under hypoxic conditions. Activation of Stat5 by EPO signaling results in the production of the anti-apoptotic bcl family member Bcl-X(L). Jak-stat pathway signaling in myocardial infarction models is associated with resistance to apoptosis. (Xuan et al. (2001) Proc Natl Acad Sci USA 98:9050-9055.) Constitutive activation of the Jak-Stat pathway results in high expression of the anti-apoptotic bcl2 family member Bcl2 and low expression of the pro-apoptotic bcl family member bax (Nielsen et al. (1999) Leukemia 13(5):735-738).

Therefore, it is known in the art that stabilization of HIFα under limited hypoxic conditions correlates with protection against apoptosis in cells exposed to oxidative stress, serum withdrawal, and chemical stress. Compounds of the invention have been shown to induce expression of glycolytic factors, and to increase expression of various factors, e.g., VEGF and EPO, which appear to act, at least in certain contexts, in a cytoprotective capacity. Compounds of the invention have also been shown to reduce infarct size, e.g., following myocardial infarction (data not shown). (See, e.g., International Application No. PCT/US 03/38689, International Publication No. WO 03/053997, and International Publication No. WO 03/049689, each of which is incorporated herein by reference in its entirety.)

The present invention establishes that compounds of the invention can further be used to coordinately increase expression of cytoprotective factors, including, e.g., anti-apoptotic factors, such as HO-1, HSP70, and adrenomedullin; to decrease expression of pro-apoptotic factors, e.g., caspase-3 and caspase-7; to increase energy preservation, e.g., ATP preservation; to increase resistance to oxidative damage; to enhance anaerobic respiration; and to reduce oxygen consumption. The compounds of the present invention thus demonstrated coordinated induction of multiple cytoprotective effects, and successfully conferred cytoprotection as measured, e.g., by prevention of apoptosis as demonstrated through reduced annexin V immunostaining. The compounds specifically reduced apoptosis in cells stressed, e.g., by various oxidative toxins and bioactive cytokines. As provided herein, the methods and compounds of the present invention induce a coordinated cytoprotective response that prevents apoptosis and increases or maintains cell viability.

The compounds and methods of the present invention further demonstrate that through inhibition of HIF hydroxylase activity, aspects of this endogenous protective response can be induced to provide cytoprotective effects through the coordinated induction of multiple mechanisms including anti-apoptotic, anti-oxidant, and other protective factors, including those relevant to glycolytic shift and neovascularization activities. The present methods and compounds can be applied to achieve coordinated induction of cytoprotective effects under any conditions; including in response to a stress, to ameliorate the stress-induced consequences. Methods and compounds of the present invention are further useful in the absence of stress under conditions in which it might be desirable, for example, in anticipation of a stress, e.g., pretreatment, preconditioning, etc., prior to surgery, therapies, exposure to certain environmental conditions, etc. It is specifically contemplated that the efficacy of the present methods and compounds is not limited to efficacy under hypoxic or impaired oxygen conditions, e.g., the present methods and compounds can be used effectively to treat or pre-treat a subject under normal oxygen conditions as well under conditions in which the subject is exposed to a stress such as hypoxia.

Methods and Compounds

Various methods are provided herein, and comprise administering to a subject a compound that inhibits HIF hydroxylase activity.

A compound of the invention is thus any compound that reduces or otherwise modulates the activity of an enzyme that hydroxylates at least one amino acid residue on HIFα. In preferred embodiments, the compound inhibits HIF hydroxylase activity, thereby inhibiting the hydroxylation of at least one HIFα amino acid residue, e.g., a proline residue, an asparagine residue, an arginine residue, etc. In a preferred embodiment, the residue is a proline residue. In specific embodiments, the residue can be the P₅₆₄ residue in HIF-1α or a homologous proline in another HIFα isoform, or the P₄₀₂ residue in HIF-1α or a homologous proline in another HIFα isoform, etc. In other embodiments, the present methods may encompass inhibiting hydroxylation of at least one HIFα asparagine residue, e.g., the N₈₀₃ residue of HIF-1α or a homologous asparagine residue in another HIFα isoform. Compounds that can be used in the methods of the invention include, for example, iron chelators, 2-oxoglutarate mimetics, and modified amino acid, e.g., proline analogs, etc.

In some embodiments, the methods and compounds of the present invention inhibit HIF hydroyxlase activity by inhibiting the activity of at least one 2-oxoglutarate dioxygenase family. In a preferred embodiment, the HIF hydroxylase is selected from the group consisting of EGLN-1, EGLN-2, EGLN-3, or an enzymatically active fragment thereof.

Exemplary compounds of the present invention are disclosed in, e.g., International Publication No. WO 03/049686 and International Publication No. WO 03/053997, incorporated herein by reference in their entireties. Specifically, compounds of the invention include, but are not limited, for example, to phenanthrolines including those described in U.S. Pat. No. 5,916,898; U.S. Pat. No. 6,200,974; and International Publication No. WO 99/21860; heterocyclic carbonyl glycines including, but not limited to, substituted quinoline-2-carboxamides and esters thereof as described, e.g., in U.S. Pat. Nos. 5,719,164 and 5,726,305; substituted isoquinoline-3-carboxamides and esters thereof as described, e.g., in U.S. Pat. No. 6,093,730; and N-substituted arylsulfonylamino hydroxamic acids as described, e.g., in International Publication No. WO 00/50390. All compounds listed in these patents, in particular, those compounds listed in the compound claims and the final products of the working examples, are hereby incorporated into the present application by reference herein. Exemplary compounds from each group are 4-Oxo-1,4-dihydro-[1,10]phenanthroline-3-carboxylic acid (Compound A), [(4-Hydroxy-7-phenoxy-isoquinoline-3-carbonyl)-amino]-acetic acid (Compound G), and 3-{[4-(3,3-dibenzyl-ureido)-benzenesulfonyl]-[2-(4-methoxy-phenyl)-ethyl]-amino}-N-hydroxy-propionamide (Compound B), respectively.

Preferred compounds of the present invention include, e.g., heterocyclic carboxamides. Specifically preferred heterocyclic carboxamides include, e.g., heterocyclic carboxamides wherein the heterocycle is selected from isoquinoline, quinoline, pyridine, cinnoline, carboline, etc. Additional structural classes of preferred compounds include anthraquinones, azafluorenes, azaphenanthrolines, benzimidazoles, benzofurans, benzopyrans, benzothiophenes, catechols, chromanones, α-diketones, furans, N-hydroxyamides, N-hydroxyureas, imidazoles, indazoles, indoles, isothiadiazoles, isothiazoles, isoxadiazoles, isoxazoles, α-keto acids, α-keto amides, α-keto esters, α-keto imines, oxadiazoles, oxalyl amides, oxazoles, oxazolines, purines, pyrans, ppyrazines, pyrazoles, pyrazolines, pyridazines, pyridines, quinazolines, phenanthrolines, tetrazoles, thiadiazoles, thiazoles, thiazolines, thiophenes, and triazoles. Exemplary compounds include [(4-Hydroxy-7-phenylsulfanyl-isoquinoline-3-carbonyl)-amino]-acetic acid (Compound C), [(4-Hydroxy-6-phenylsulfanyl-isoquinoline-3-carbonyl)-amino]-acetic acid (Compound D), [(1-Chloro-4-hydroxy-7-phenoxy-isoquinoline-3-carbonyl)-amino]-acetic acid (Compound E), [(1-Bromo-4-hydroxy-7-phenoxy-isoquinoline-3-carbonyl)-amino]-acetic acid (Compound F), and [(1-Chloro-4-hydroxy-isoquinoline-3-carbonyl)-amino]-acetic acid (Compound H).

In preferred embodiments, the compounds of the invention inhibit HIF hydroxylase activity by inhibiting HIF prolyl hydroxylase activity. A HIF prolyl hydroxylase or HIF-PH is any enzyme that is capable of hydroxylating a proline residue in the HIF protein. Preferably, the proline residue hydroxylated by HIF-PH includes the proline found within the motif LXXLAP, e.g., as occurs in the human HIF-1α native sequence at L₃₉₇TLLAP and L₅₅₉EMLAP. HIF-PH includes members of the Egl-Nine (EGLN) gene family described by Taylor (2001, Gene 275:125-132), and characterized by Aravind and Koonin (2001, Genome Biol 2:RESEARCH0007), Epstein et al. (2001, Cell 107:43-54), and Bruick and McKnight (2001, Science 294:1337-1340). Examples of HIF-PH enzymes include human SM-20 (EGLN1) (GenBank Accession No. AAG33965; Dupuy et al. (2000) Genomics 69:348-54), EGLN2 isoform 1 (GenBank Accession No. CAC42510; Taylor, supra), EGLN2 isoform 2 (GenBank Accession No. NP_{—}060025), and EGLN3 (GenBank Accession No. CAC42511; Taylor, supra); mouse EGLN1 (GenBank Accession No. CAC42515), EGLN2 (GenBank Accession No. CAC42511), and EGLN3 (SM-20) (GenBank Accession No. CAC42517); and rat SM-20 (GenBank Accession No. AAA19321). Additionally, HIF-PH may include Caenorhabditis elegans EGL-9 (GenBank Accession No. AAD56365) and Drosophila melanogaster CG1114 gene product (GenBank Accession No. AAF52050). HIF-PH also includes any active fragment of the foregoing full-length proteins.

Methods for identifying compounds of the invention are also provided. In certain aspects, a compound of the invention is one that inhibits HIF hydroxylase activity. Assays for hydroxylase activity are standard in the art. Such assays can directly or indirectly measure hydroxylase activity. For example, an assay can measure hydroxylated residues, e.g., proline, asparagine, etc., present in the enzyme substrate, e.g., a target protein, a synthetic peptide mimetic, or a fragment thereof. (See, e.g., Palmerini et al. (1985) J Chromatogr 339:285-292.) A reduction in hydroxylated residue, e.g., proline or asparagine, in the presence of a compound is indicative of a compound that inhibits hydroxylase activity. Alternatively, assays can measure other products of the hydroxylation reaction, e.g., formation of succinate from 2-oxoglutarate. (See, e.g., Cunliffe et al. (1986) Biochem J 240:617-619.) Kaule and Gunzler (1990; Anal Biochem 184:291-297) describe an exemplary procedure that measures production of succinate from 2-oxoglutarate.

Procedures such as those described above can be used to identify compounds that modulate HIF hydroxylase activity. Target protein may include HIFα or a fragment thereof, e.g., HIF(556-575). Enzyme may include a HIF prolyl hydroxylase (e.g., GenBank Accession No. AAG33965, etc.), or a HIF asparaginyl hydroxylase (e.g., GenBank Accession No. AAL27308, etc.), etc., or an active fragment thereof, obtained from any source. Enzyme may also be present in a crude cell lysate or in a partially purified form. For example, procedures that measure HIF hydroxylase activity are described in Ivan et al. (2001, Science 292:464-468; and 2002, Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIFα.

A compound of the invention is one that confers cytoprotection as measured, for example, by reduced annnexin V staining. In certain aspects, a compound of the invention produces a measurable effect, as measured in vitro or in vivo, as demonstrated by a measurable indication of induction of a cytoprotective effect. This can include, for example, a demonstrated increase in expression of cytoprotective factors, e.g., adrenomedullin, caspace 3, caspace 7, HO-1, HSP-70, VEGF, EPO, various glycolytic factors, etc. Such measurements can be assayed, e.g., using methods available in the art and those described herein by way of example.

Pharmaceutical Formulations and Routes of Administration

The compositions of the present invention can be delivered directly or in pharmaceutical compositions containing excipients, as is well known in the art. Present methods of treatment can comprise administration of an effective amount of a compound of the present invention to a subject. In various embodiments, the subject is a cell, a population of cells, a tissue, an organ, or an organism. In certain embodiments, the subject is an animal, a mammal, and, most preferably, a human subject.

An effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons dervided from methan and ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal, or human that is being sought by the researcher, veterinarian, medical doctor, or other clinician, e.g., increased cell viability, decrease or prevention of apoptosis, increased expressioin of anti-apoptotic factors, decreased expression of pro-apoptotic factors, increased energy preservation, prevention of oxidative damage, etc.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

The compounds of the invention are compounds that inhibit HIF hydroxylase activity. In one embodiment, the compound of the invention is selected from the group consisting of phenanthrolines; heterocyclic carbonyl glycines including, but not limited to, substituted quinoline-2-carboxamides and isoquinoline-3-carboxamides; and N-substituted arylsulfonylamino hydroxamic acids. In preferred embodiments, the compound of the invention is selected from the group consisting of 4-Oxo-1,4-dihydro-[1,10]phenanthroline-3-carboxylic acid (Compound A), 3-{[4-(3,3-Dibenzyl-ureido)-benzenesulfonyl]-[2-(4-methoxy-phenyl)-ethyl]-amino}-N-hydroxy-propionamide (Compound B), [(4-Hydroxy-7-phenylsulfanyl-isoquinoline-3-carbonyl)-amino]-acetic acid (Compound C), [(4-Hydroxy-6-phenylsulfanyl-isoquinoline-3-carbonyl)-amino]-acetic acid (Compound D), [(1-Chloro-4-hydroxy-7-phenoxy-isoquinoline-3-carbonyl)-amino]-acetic acid (Compound E), [(1-Bromo-4-hydroxy-7-phenoxy-isoquinoline-3-carbonyl)-amino]-acetic acid (Compound F), [(4-Hydroxy-7-phenoxy-isoquinoline-3-carbonyl)-amino]-acetic acid (Compound G), and [(1-Chloro-4-hydroxy-isoquinoline-3-carbonyl)-amino]-acetic acid (Compound H).

### Example 1

### Increased Adrenomedullin Gene Expression

Adrenomedullin (ADM), a hypotensive peptide highly expressed in several tissues, including adrenal medulla, cardiac ventricle, lung, and kidney, has been associated with cytoprotective effects. For example, treatment of retinal pigment epithelial cells with ADM ameliorated a hypoxia-induced decrease in cell number. (Udono et al. (2001) Invest Ophthal Vis Sci 42:1080-1086.) Compounds and methods of the present invention were tested for induction of adrenomedullin in various cell types as follows.

Hep3B cells (ATCC No. HB-8064) were grown in DMEM containing 8% fetal bovine serum. Hep3B cells were seeded into 6-well culture dishes at ˜500,000 cells per well. After 8 hours, the media was changed to DMEM containing 0.5% fetal bovine serum and the cells were incubated for an additional 16 hours. Compound A, compound B, compound C, compound G, or compound H was added to the cells (25 μM final concentration) and the cells were incubated for various times. Control cells were incubated with vehicle (DMSO) with no compound treatment. Harvested cells were assessed for cell viability (GUAVA), or added to RNA extraction buffer (RNeasy, Qiagen) and stored at −20° C. for subsequent RNA purification.

RNA was precipitated in 0.3 M sodium acetate (pH 5.2), 50 ng/ml glycogen, and 2.5 volumes of ethanol for one hour at −20° C. Samples were centrifuged and pellets were washed with cold 80% ethanol, dried, and resuspend in water. Double stranded cDNA was synthesized using a T7-(dT)24 first strand primer (Affymetrix, Inc., Santa Clara Calif.) and the SUPERSCRIPT CHOICE system (Invitrogen) according to the manufacturer's instructions. The final cDNA was extracted with an equal volume of 25:24:1 phenol:chloroform:isoamyl alcohol using a PHASE LOCK GEL insert (Brinkman, Inc., Westbury N.Y.). The aqueous phase was collected and cDNA was precipitated using 0.5 volumes of 7.5 M ammonium acetate and 2.5 volumes of ethanol. Alternatively, cDNA was purified using the GENECHIP sample cleanup module (Affymetrix) according to the manufacturer's instructions.

Biotin-labeled cRNA was synthesized from the cDNA in an in vitro translation (IVT) reaction using a BIOARRAY HighYield RNA transcript labeling kit (Enzo Diagnostics, Inc., Farmingdale N.Y.) according to the manufacturer's instructions. Final labeled product was purified and fragmented using the GENECHIP sample cleanup module (Affymetrix) according to the manufacturer's instructions.

Hybridization cocktail was prepared by bringing 5 μg probe to 100 μl in 1× hybridization buffer (100 mM MES, 1 M [Na⁺], 20 mM EDTA, 0.01% Tween 20), 100 μg/ml herring sperm DNA, 500 μg/ml acetylated BSA, 0.03 nM contol oligo B2 (Affymetrix), and 1× GENECHIP eukaryotic hybridization control (Affymetrix). The cocktail was sequentially incubated at 99° C. for 5 minutes and 45° C. for 5 minutes, and then centrifuged for 5 minutes. The Murine genome MOE430Aplus2 array (Affymetrix) was brought to room temperature and then prehybridized with 1× hybridization buffer at 45° C. for 10 minutes with rotation. The buffer was then replaced with 80 μl hybridization cocktail and the array was hybridized for 16 hours at 45° C. at 60 rpm with counter balance. Following hybridization, arrays were washed once with 6×SSPE, 0.1% Tween 20, and then washed and stained using R-phycoerythrin-conjugated streptavidin (Molecular Probes, Eugene Oreg.), goat anti-streptavidin antibody (Vector Laboratories, Burlingame Calif.), and a GENECHIP Fluidics Station 400 instrument (Affymetrix) according to the manufacturer's EukGE-WS2v4 protocol (Affymetrix). Arrays were analyzed using a GENEARRAY scanner (Affymetrix) and Microarray Suite software (Affymetrix).

RNA quality was monitored by capillary electrophoresis (Agilent Bioanalyzer). Hybridization cocktails were prepared as described (Affymetrix), and hybridized to Affymetrix human U133A arrays containing 22,283 probe sets. The Human Genome U133A array (Affymetrix) represents all sequences in the Human Unigene database build 133 (National Center for Biotechnology Information, Bethesda Md.), including approximately 14,500 well-characterized human genes. Array performance was analyzed with Affymetrix MicroArray Suite (MAS) software and individual probe sets were assigned “present”, “marginal, and “absent” calls according to software defaults. Statistical analyses and filtered probe set lists were prepared using GeneSpring software (Silicon Genetics). Cutoffs for “expressed” probe sets used a combination of Affymetrix “P” calls and absolute expression values derived from Genespring's intrinsic data error model. Data was normalized to averaged control samples.

Replicate microarrays were probed using RNA isolated from replicate experiments conducted on different days. Data is reported as an average of these two determinations.

Expression of the gene encoding adrenomedullin, represented on the microarray, was specifically analyzed. Results shown in Table 1 below are presented as fold-increase in adrenomedullin gene expression above non-treated control.

TABLE 1

| Compound | Time | Adrenomedullin |
|---|---|---|
| A | 6 hrs | 3.582 |
| B | 6 hrs | 8.334 |
| H | 6 hrs | 1.298 |
| C | 6 hrs | 3.896 |
| G | 6 hrs | 3.278 |

As shown above in Table 1, addition of various compounds of the present invention increased expression of the gene encoding adrenomedullin. Increased expression of adrenomedullin by compounds of the present invention was rapid, occurring within at least 6 hours after compound addition. Additionally, expression of the gene encoding adrenomedullin remained elevated and continued to increase over 48 hours following compound addition.

Adrenomedullin gene expression following compound treatment was also examined in peripheral blood mononuclear cells (PBMCs). Whole human blood was collected and processed immediately. The blood was diluted with an equal volume of phosphate buffered saline. FICOLL-PAQUE PLUS (Amersham Biosciences) was layered under the blood and the tubes were centrifuged at 350×g for 12 minutes at room temperature. PBMCs formed a visible layer in the middle layer of the tube. PBMCs were carefully removed from the tube, diluted with 3 volumes of phosphate buffered saline, and pelleted by centrifugation for 5 minutes at 300×g at room temperature. PBMCs were cultured in DMEM containing 2.5% fetal bovine serum and treated with either 0.25% DMSO or compound G (5 μM) in 0.25% DMSO for 20 hours. The cells were then pelleted and stored at −20° C. in RLT lysis buffer (Qiagen Inc., Valencia, Calif.) containing 1% beta-mercaptoethanol. Total RNA was isolated using the RNeasy kit (Valencia, Calif.).

PBMCs treated with compound G showed greater than 3.518-fold increase in expression of the gene encoding adrenomedullin compared to non-treated control cells.

Adrenomedullin gene expression was also examined in cardiomyocytes treated with compound and subsequently challenged with KCN, an inhibitor of oxidative glucose metabolism. H9c2 rat cardiomyocytes were cultured in 96-well tissue culture plates (approximately 20,000 cells per well) in DMEM containing 10% fetal bovine serum. Media was changed to DMEM containing 0.5% fetal bovine serum, and the cells were treated with 10 μM compound C or compound D for 24 hours. Media was then replaced with glucose-free DMEM (Gibco/Invitrogen Cat. # 11966-025) containing 2 mM KCN (Sigma-Aldrich Cat. No. 207810). Results of adrenomedullin gene expression, presented as fold-increase in gene expression above DMSO control, are shown below in Table 2.

TABLE 2

| Treatment | KCN | Adm |
|---|---|---|
| DMSO control | No | 1.0 |
| Cmpd. C | No | 4.8 |
| Cmpd. D | No | 2.2 |
| DMSO control | Yes | 0.9 |
| Cmpd. C | Yes | 7.9 |
| Cmpd. D | Yes | 4.2 |

As shown above in Table 2, treatment of cardiomyocytes with compound C or compound D increased adrenomedullin gene expression above that observed in control cultures. Cells treated with KCN, also had increased adrenomedullin gene expression following treatment with compound C or compound D.

Taken together, these results indicated that methods and compounds of the present invention increased expression of adrenomedullin, a protein associated with anti-apoptotic and anti-oxidant effects, in various cells. Induction of adrenomedullin by the compounds and methods disclosed herein demonstrate cytoprotective aspects of the present invention. Further, the induction of cytoprotective factors, including adrenomedullin, in cells under stress, e.g., hypoxic stress (Udono et al., supra) or KCN-induced metabolic stress, demonstrate a cytoprotective response in various cells using the present methods.

### Example 2

### Decreased Caspase Activity

The apoptosis-related cysteine proteases, e.g., caspase-3 and caspase-7, are directly involved in cell apoptosis. Activation of cyclin-dependent kinase (CDK)-2 through caspase-mediated cleavage of CDK inhibitors is instrumental in the execution of apoptosis following caspase activation. (Levkau et al. (1998) Molec Cell 1:553-563.) Apoptosis, therefore, is associated with increased levels of caspases and caspase activity. The cytoprotective effects of the methods and compounds of the present invention were thus tested for their effect on caspase-mediated apoptosis in cells as follows.

SH-SY5Y cells (human neuroblastoma cells) were plated in 96-well culture plates at 60,000 cells per well. Following overnight incubation, cells were washed one time with DMEM containing 1% fetal bovine serum and cultured in identical media with either vehicle control (DMSO) or 20 μM compound G in a total volume of 200 μl per well. After 24 hours, cells were washed with serum-free media and then incubated with DMEM containing 10% fetal bovine serum with either vehicle control (DMSO) or 20 μM compound G in a total volume of 200 μl per well. Replicate cultures received DMEM with 1% fetal bovine serum and were cultured with either vehicle control (DMSO) or 20 μM compound G, in the absence or presence of 500 nM staurosporin, a kinase inhibitor that induces cellular apoptosis by a caspase-dependent mechanism. (Jacobsen et al. (1996) J Cell Biol. 133:1041-51.). After an additional 24 hour incubation, caspase activity was assayed using a caspase-3 and caspase-7 fluorometric assay according to the manufacturer's instructions (Apo-ONE Homogenous Caspase 3/7 Assay, Promega, Wis.).

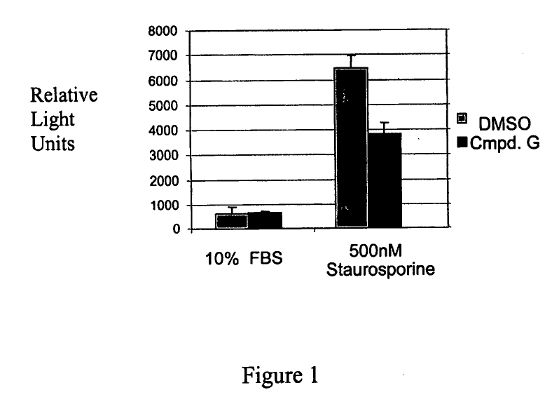

As shown in FIG. 1, caspase activity was increased in SH-SY5Y cells that were cultured with staurosporine, but significantly inhibited if cells were pretreated with compound G prior to staurosporine treatment. Treatment of cells with DMSO or compound G showed no differences in caspase activity in the presence of 10% fetal bovine serum. The results showed that treatment of cells with compound of the present invention prior to challenge with staurosporine reduced caspase activity/levels. These results indicated that compound of the present invention reduced caspase-mediated apoptosis and thus provided cytoprotection to the cells. The lack of any effect on caspase activity in cells not undergoing apoptosis, i.e. cells cultured in 10% fetal bovine serum, shows that compound G specifically inhibited caspase-mediated apoptosis in response to staurosporine addition, and that compound G was not a direct caspase inhibitor.

### Example 3

### ATP Preservation

Metabolic challenge, e.g., by inducing oxidative stress or inhibiting oxidative metabolism, compromises cell viability by rapidly depleting ATP stores in metabolically active cells. In one aspect, cytoprotection requires adequate production and/or preservation of ATP in the cell to meet the ongoing demands of maintaining cell structure and function. To demonstrate the ability of the compounds and methods of the present invention to preserve ATP levels in challenged cells, the following experiment was performed.

H9c2 rat cardiomyocytes were incubated with 10 mM homocysteic acid (HCA) in the absence or presence of various concentrations of compound G as indicated for 24 hours. Cell viability was determined by measuring intracellular ATP levels. Quantitation of intracellular ATP levels was performed using the ViaLight Plus™ kit (Cambrex Cat. No. LT17-221) according to the manufacturers instructions.

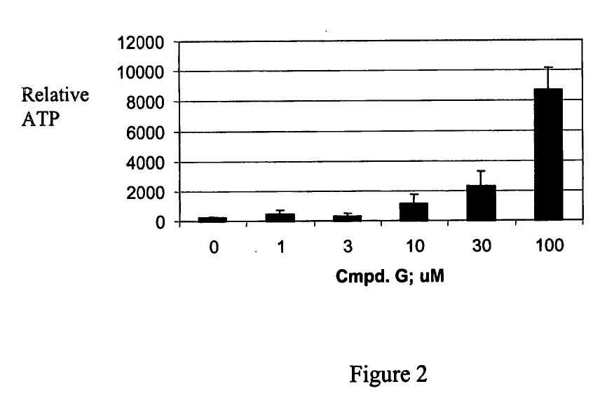

HCA induces glutathione depletion in cells, thereby decreasing the reducing capacity of cells. Therefore, cells treated with HCA are under oxidative stress. As shown in FIG. 2, treatment of cells with compound G as compared to vehicle control (DMSO) in the presence of HCA resulted in dose-dependent increases in intracellular ATP levels. Intracellular ATP levels were unchanged in cells not incubated with HCA (data not shown).

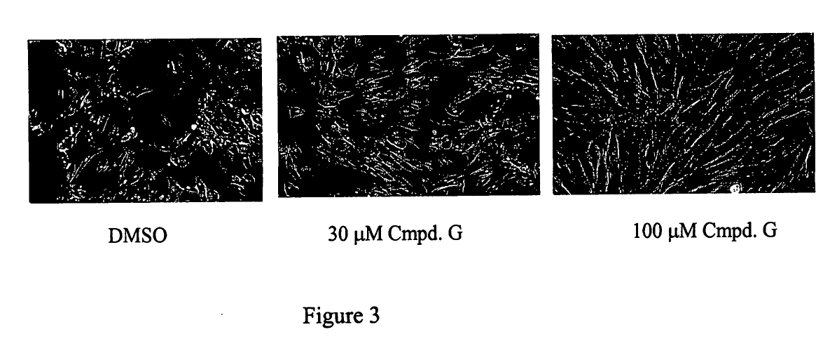

Phase contrast microscopy of cells treated with HCA correlated with the results shown for intracellular ATP levels in vehicle (DMSO) and compound G treated cells. Thus, cells exposed to HCA and subsequently treated with vehicle were sparse and showed a rounded morphology, indicative of low cell viability in response to oxidative stress. (See FIG. 3.) In contrast, cardiomyocytes treated with HCA in the presence of 30 μM compound G were still confluent, appeared less rounded, and maintained a morphology consistent with viable cardiomyoctes. These results showed that compounds and methods of the present invention are useful for maintaining cell viability under conditions of oxidative stress. Additionally, these results showed that methods and compounds of the present invention preserve intracellular ATP levels, useful and required for maintaining basal metabolic processes and cell viability. The results also indicated that methods and compounds of the present invention provide cytoprotection to cells under oxidative stress.

In similar experiments, H9c2 rat cardiomyocytes were pretreated with either vehicle control (0.5% DMSO) or 20 μM of Compound A, Compound B, Compound C, Compound D, Compound E, Compound F, Compound G, or compound H. After 24 hours, cells were washed with serum-free DMEM and subjected to oxygen and glucose deprivation by incubating the cells in glucose-free DMEM and 2 mM KCN for 30 minutes. Intracellular ATP levels were then determined. Table 3 below shows ATP levels (represented as relative light units) in cells treated with various compounds of the present invention.

TABLE 3

| Compound | ATP (relative light units) |
|---|---|
| A | 968.5 |
| B | 683.5 |
| C | 3710 |
| D | 859.5 |
| E | 2043 |
| F | 1134 |
| G | 2128.3 |
| H | 947 |
| Vehicle control (DMSO) | 415 |

Cells deprived of nutrients (i.e., glucose) and oxygen (i.e., inhibition of oxidative respiration) showed a dramatic and rapid decrease in intracellular ATP levels. Cells treated with compound of the present invention prior to deprivation of nutrients and oxygen showed higher levels of intracellular ATP than non-treated control cells. These results indicated that methods and compounds of the present invention are effective at preserving intracellular ATP levels in cells exposed to stress, such as low-glucose or decreased oxidative respiration. The data also suggested that treatment of cells, tissues, and organs with a compound of the present invention is effective for inducing cytoprotection or cytoprotective events prior to a condition of stress.

### Example 4

### Increase Heme Oxygenase-1 Gene Expression

Heme oxygenase (HO)-1 is known to exert various cytoprotective mechanisms offering anti-apoptotic, anti-oxidant, and anti-inflammatory effects. The following experiment was performed to demonstrate that the methods and compounds of the present invention regulate expression of heme oxygenase, and thereby induce its cytoprotective effects.

Rat H9c2 cardiomyocytes were treated with either vehicle control (DMSO) or with 10 μM of Compound C or Compound D. After 24 hours, cells were harvested and RNA isolated for analysis of HO-1 gene expression by microarray analysis. Total RNA was isolated from cells using the RNeasy kit (Qiagen), and prepared for microarray analysis as described above in Example 1. Microarray analysis was performed using the Murine Genome MOE430Aplus2 array (Affymetrix) represents all sequences in the Murine UniGene database build 107 (National Center for Biotechnology Information, Bethesda Md.), including approximately 14,000 well-characterized mouse genes.

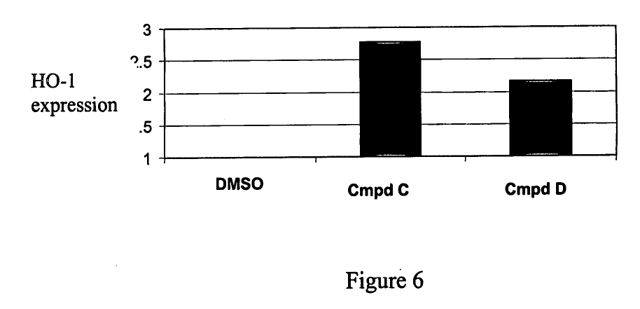

As shown in FIG. 6, treatment of cells with either compound C or compound D increased HO-1 gene expression 2- to 3-fold in cardiomyocytes compared to that observed in non-treated control cells.

In another series of experiments, H9c2 rat cardiomyocytes were treated with either vehicle control or with various concentrations (1 μM, 3 μM, 10 μM, 30 μM, and 100 μM) of compound C or compound G. Cell lysates were harvested and HO-1 protein levels determined by ELISA according to the manufacturer's instructions (cat # EKS-810; Stressgen, Victoria, BC, Canada). Data shown below in Table 4 represents values obtain with 100 μM compound.

TABLE 4

| | HO-1 |
|---|---|
| Compound | (ng/mg total cell protein) |
| C | 102.68 |
| G | 28.54 |
| control | 2.86 |

As shown in Table 4 above, compounds of the present invention increased expression of HO-1 protein in cardiomyocytes in a dose-dependent fashion. Increased HO-1 proteins levels were observed in cells treated with various compounds of the present invention. The results indicated that methods and compounds of the present invention are useful for increasing expression of HO-1 mRNA and protein in cells and tissues. Since HO-1 has been shown to be cytoprotective for cells and tissues exposed to stress, e.g. ischemia, compounds and methods of the present invention provide cytoprotective effects on cells and tissues by, for example, increasing expression of HO-1.

### Example 5

### Increased HSP70 Gene Expression

Similar to ADM and HO-1, the expression of heat shock protein (Hsp)70 has been associated with cytoprotective effects in numerous systems. (See, e.g., Zhu et al. (2003) Arterioscler Thromb Vasc Biol 23(6): 1055-1059; Mestril et al. (1994) J Clin Invest 93(2):759-756; Heads et al. (1995) J Mol Cell Cardiol 27(8):1669-1678.) To demonstrate induction of Hsp70 using the current compounds and methods, the following experiments were performed.

Animal Dosing Study I

Twelve Swiss Webster male mice (30-32 g) were obtained from Simonsen, Inc (Gilroy, Calif.) and treated by oral gavage two times per day for 2.5 days (5 doses) with a 4 ml/kg volume of either 0.5% carboxymethyl cellulose (CMC; Sigma-Aldrich, St. Louis Mo.) (0 mg/kg/day) or 2.5% compound H (25 mg/ml in 0.5% CMC) (200 mg/kg/day). Four hours after the final dose, the mice were then sacrificed and approximately 150 mg of liver and each kidney were isolated and stored in RNALATER solution (Ambion) at −20° C.

Animal Dosing Study III

To determine gene induction patterns over time, twenty four Swiss Webster male mice (30-32 g) were obtained from Simonsen, Inc. and treated by oral gavage with a 4 ml/kg volume of either 0.5% carboxymethyl cellulose (CMC; Sigma-Aldrich, St. Louis Mo.) (0 mg/kg) or 1.25% compound H (25 mg/ml in 0.5% CMC) (100 mg/kg). At 4, 8, 16, 24, 48, or 72 hours after the final dose, animals were anesthetized with isoflurane. The mice were then sacrificed and tissue samples of kidney, liver, brain, lung, and heart were isolated and stored in RNALATER solution (Ambion) at −80° C. RNA isolation and gene expression analysis were performed as described below.

RNA isolation was carried out using the following protocol. A section of each organ was diced, 875 μl of RLT buffer (RNEASY kit; Qiagen Inc., Valencia Calif.) was added, and the pieces were homogenized for about 20 seconds using a rotor-stator POLYTRON homogenizer (Kinematica, Inc., Cincinnati Ohio). The homogenate was micro-centrifuged for 3 minutes to pellet insoluble material, the supernatant was transferred to a new tube and RNA was isolated using an RNEASY kit (Qiagen) according to the manufacturer's instructions. The RNA was eluted into 801 μL of water and quantitated with RIBOGREEN reagent (Molecular Probes, Eugene Oreg.). The absorbance at 260 and 280 nm was measured to determine RNA purity and concentration.

Alternatively, tissue samples were diced and homogenized in TRIZOL reagent (Invitrogen Life Technologies, Carlsbad Calif.) using a rotor-stator POLYTRON homogenizer (Kinematica). Homogenates were brought to room temperature, 0.2 volumes chloroform was added, and samples were mixed vigorously. Mixtures were incubated at room temperature for several minutes and then were centrifuged at 12,000 g for 15 min at 4° C. The aqueous phase was collected and 0.5 volumes of isopropanol were added. Samples were mixed, incubated at room temperature for 10 minutes, and centrifuged for 10 min at 12,000 g at 4° C. The supernatant was removed and the pellet was washed with 75% EtOH and centrifuged at 7,500 g for 5 min at 4° C. The absorbance at 260 and 280 nm was measured to determine RNA purity and concentration.

RNA was precipitated in 0.3 M sodium acetate (pH 5.2), 50 ng/ml glycogen, and 2.5 volumes of ethanol for one hour at −20° C. Samples were centrifuged and pellets were washed with cold 80% ethanol, dried, and resuspend in water. Double stranded cDNA was synthesized using a T7-(dT)24 first strand primer (Affymetrix, Inc., Santa Clara Calif.) and the SUPERSCRIPT CHOICE system (Invitrogen) according to the manufacturer's instructions. The final cDNA was extracted with an equal volume of 25:24:1 phenol:chloroform:isoamyl alcohol using a PHASE LOCK GEL insert (Brinkman, Inc., Westbury N.Y.). The aqueous phase was collected and cDNA was precipitated using 0.5 volumes of 7.5 M ammonium acetate and 2.5 volumes of ethanol. Alternatively, cDNA was purified using the GENECHIP sample cleanup module (Affymetrix) according to the manufacturer's instructions.

Biotin-labeled cRNA was synthesized from the cDNA in an in vitro translation (IVT) reaction using a BIOARRAY HighYield RNA transcript labeling kit (Enzo Diagnostics, Inc., Farmingdale N.Y.) according to the manufacturer's instructions. Final labeled product was purified and fragmented using the GENECHIP sample cleanup module (Affymetrix) according to the manufacturer's instructions.

Hybridization cocktail was prepared by bringing 5 μg probe to 100 μl in 1× hybridization buffer (100 mM MES, 1 M [Na⁺], 20 mM EDTA, 0.01% Tween 20), 100 μg/ml herring sperm DNA, 500 μg/ml acetylated BSA, 0.03 nM contol oligo B2 (Affymetrix), and 1× GENECHIP eukaryotic hybridization control (Affymetrix). The cocktail was sequentially incubated at 99° C. for 5 minutes and 45° C. for 5 minutes, and then centrifuged for 5 minutes. The Murine genome MOE430Aplus2 array (Affymetrix) was brought to room temperature and then prehybridized with 1× hybridization buffer at 45° C. for 10 minutes with rotation. The buffer was then replaced with 80 μL hybridization cocktail and the array was hybridized for 16 hours at 45° C. at 60 rpm with counter balance. Following hybridization, arrays were washed once with 6×SSPE, 0.1% Tween 20, and then washed and stained using R-phycoerythrin-conjugated streptavidin (Molecular Probes, Eugene Oreg.), goat anti-streptavidin antibody (Vector Laboratories, Burlingame Calif.), and a GENECHIP Fluidics Station 400 instrument (Affymetrix) according to the manufacturer's EukGE-WS2v4 protocol (Affymetrix). Arrays were analyzed using a GENEARRAY scanner (Affymetrix) and Microarray Suite software (Affymetrix).

The Murine Genome MOE430Aplus2 array (Affymetrix) represents all sequences in the Murine UniGene database build 107 (National Center for Biotechnology Information, Bethesda Md.), including approximately 14,000 well-characterized mouse genes.

As shown in Table 5 below, in vivo administration of compound H resulted in increased expression of the gene encoding HSP70-3 in mouse liver and lung. Additionally,

TABLE 5

| | HSP70-3 mRNA Levels | HSP70-3 mRNA Levels |
|---|---|---|
| Animal Study | Liver | Lung |
| III | 1.441 | 3.0 |
| I | 2.77 | ND |

These results demonstrate the compounds and methods of the present invention increase Hsp70 in cells, thus eliciting the cytoprotective benefits thereof. Taken together, the results shown in Examples 1 to 5 demonstrate a coordinated induction of cytoprotective factors using the present methods and compounds. Unlike single gene product cytoprotective effects, the present methods provide a coordinate induction of the innate cytoprotective factors and processes contained within a cell. Such induction, provided either before or subsequent to an initiating stress, can provide substantial survival benefit to individual cells, and thus to tissues and organs as a whole. Specifically, these results suggested that methods and compounds of the present invention are useful for increasing expression of genes associated with cytoprotective and anti-oxidant effects.

### Example 6

### Reduced Apoptosis

Based on the results shown in Examples 1 to 5, demonstrating the coordinated induction of cytoprotective factors, inhibition of apoptotic processes, and resulting cytoprotective effects, the effect of compounds of the present invention on preventing or decreasing apoptosis was examined. Human umbilical vein endothelial cells (HUVEC) were plated in DMEM containing 0.5% fetal bovine serum that was supplemented with 1 ng/ml of vascular endothelial growth factor. After overnight culture, the cells were washed with PBS and incubated for an additional 24 hours in DMEM containing 0.5% fetal bovine serum and either vehicle control (DMSO) or 25 μM of compound G. The cell cultures were subsequently washed and re-cultured with DMEM containing 0.5% fetal bovine serum containing 20 ng/ml TNF-α for an additional 4 or 8 hours. The cells were then harvested and immunostained with either FITC-conjugated isotype control or FITC-conjugated Annexin V for identification and determination of cells undergoing apoptosis. (Koopman et al. (1994) Blood 84:1415-1420.) Annexin V preferentially binds negatively charged phospholipids, like phosphatidylserine, which are associated with plasma membrane changes in apoptotic cells. Annexin V binding allows for the identification and quantitation of cells at early stages of apoptosis, when apoptosis occurs in the absence of DNA fragmentation, and the discrimination between cell death associated with apoptosis or with necrosis.

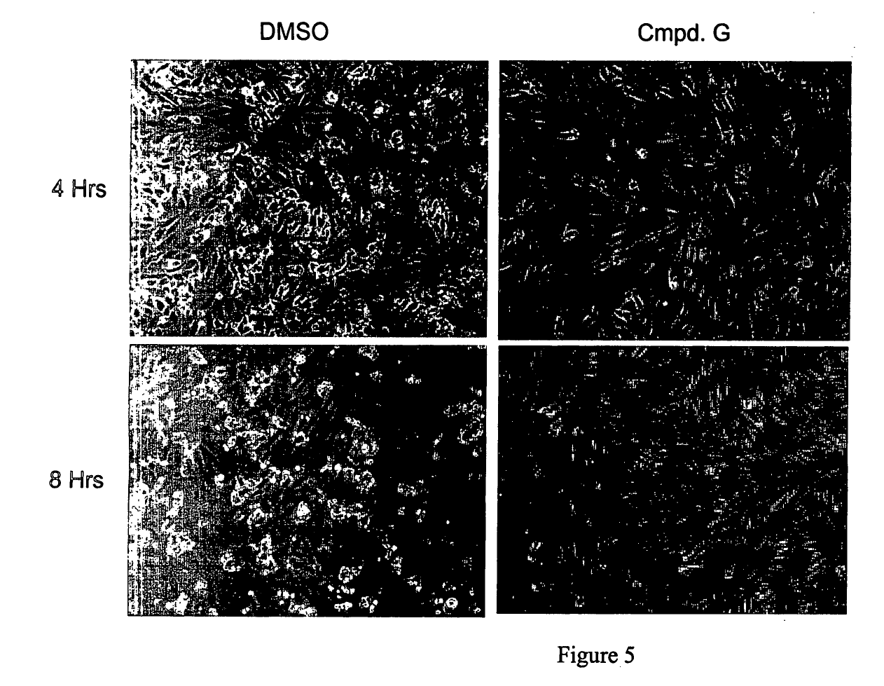

As shown in FIG. 5, TNF-α addition to HUVECs increased annexin V immunostaining, as measured by increased mean fluorescence intensity. This result indicated that apoptosis was induced in HUVECs in response to TNF-α treatment. HUVECs treated with compound G 24 hours prior to addition of TNF-α (for 4 or 8 hours) had reduced annexin V immunostaining compared to cells treated with TNF-α in the absence of compound G. (See FIG. 5.) Annexin V levels, as determined by mean fluorescence intensity, in cells treated with TNF-α and compound G were essentially the same as that observed in control cells treated with DMSO alone. These results indicated that compounds and methods of the present invention prevented TNF-α induced apoptosis in HUVECs.

In replicate HUVEC cultures treated as described above, light micrographs were taken of cells treated with vehicle control (DMSO) and compound G following stimulation with 20 ng/ml TNF-α for 4 and 8 hours (FIG. 6). At both time points, HUVECs treated with DMSO and stimulated with TNF-α exhibited a pro-apoptotic morphology, consistent with the Annexin V immunostaining results described above. HUVECs displaying a pro-apoptotic morphology were characterized by having rounded morphology and by showing signs of detaching from the tissue culture plate. (See FIG. 6.) HUVECs treated with compound G prior to treatment with TNF-α exhibited a viable and normal morphology (FIG. 6), similar to that observed in cells not treated with TNF-α (data not shown).

Together, these results indicated that treatment of HUVECs with TNF-α induced cell surface marker expression (i.e., Annexin V) and changes in cellular morphology consistent with cells undergoing apoptosis. Treatment of cells with compound G for 24 hours prior to TNF-α treatment inhibited the increase in Annexin V and resulted in cells maintaining a viable morphology and phenotype. Thus, methods and compounds of the present invention are cytoprotective to cells undergoing stress responses that induce apoptosis, as shown here, e.g. by treatment of HUVECs with TNF-α.

Various modifications of the invention, in addition to those shown and described herein, will become apparent to those skilled in the art from the foregoing description. Such modifications are intended to fall within the scope of the appended claims.

All references cited herein are hereby incorporated herein by reference in their entirety.

What is claimed is:

1. A method for conferring cytoprotection on a cell, the method comprising administering to the cell an effective amount of a compound that inhibits HIF hydroxylase activity, thereby conferring cytoprotection on the cell.

2. A method for inducing a cytoprotective effect in a cell, the method comprising administering to the cell an effective amount of a compound that inhibits HIF hydroxylase activity, thereby inducing the cytoprotective effect in the cell.

3. The method of any of claims 1 and 2, wherein the administering is in vitro.

4. The method of any of claims 1 and 2, wherein the administering is in vivo.

5. The method of claim 2, wherein the cytoprotective effect is selected from the group consisting of increased energy preservation, increased ATP preservation, increased anaerobic respiration, reduced oxygen consumption, reduced oxidative damage, increased expression of at least one factor having anti-oxidant activity, prevention or reduction of apoptosis, increased expression of at least one anti-apoptotic factor, decreased expression of at least one pro-apoptotic factor, and increased expression of at least one cytoprotective factor.

6. The method of claim 5, wherein the factor having anti-oxidant activity is selected from the group consisting of adrenomedullin, heme oxygenase-1, and HSP70.

7. The method of claim 5, wherein the anti-apoptotic factor is selected from the group consisting of adrenomedullin, heme oxygenase-1, and HSP70.

8. The method of claim 5, wherein the pro-apoptotic factor is selected from the group consisting caspase-3 and caspase-7.

9. The method of claim 5, wherein the cytoprotective factors are selected from the group consisting of erythropoietin and vascular endothelial cell growth factor.

10. A method for increasing adrenomedullin expression in a cell, the method comprising administering to the cell an effective amount of a compound that inhibits HIF hydroxylase activity, thereby increasing adrenomedullin expression in the cell.

11. A method for increasing HSP70 expression in a cell, the method comprising administering to the cell an effective amount of a compound that inhibits HIF hydroxylase activity, thereby increasing HSP70 expression in the cell.

12. A method for increasing heme oxygenase-1 expression in a cell, the method comprising administering to the cell an effective amount of a compound that inhibits HIF hydroxylase activity, thereby increasing heme oxygenase-1 expression in the cell.

13. A method for decreasing caspase expression in a cell, the method comprising administering to the cell an effective amount of a compound that inhibits HIF hydroxylase activity, thereby decreasing caspase expression in the cell.

14. The method of claim 13, wherein the caspase is selected from the group consisting of caspase-3 and caspase-7.

15. A method for preserving ATP levels in a cell, the method comprising administering to the cell an effective amount of a compound that inhibits HIF hydroxylase activity, thereby preserving ATP levels in the cell.

16. A method for reducing or preventing apoptosis in a cell, the method comprising administering to the cell an effective amount of a compound that inhibits HIF hydroxylase activity, thereby reducing or preventing apoptosis in the cell.

17. A method for increasing expression of an anti-apoptotic factor in a cell, the method comprising administering to the cell an effective amount of a compound that inhibits HIF hydroxylase activity, thereby increasing expression of the anti-apoptotic factor in the cell.

18. The method of claim 17, wherein the anti-apoptotic factor is selected from the group consisting of adrenomedullin, heme oxygenase-1, and HSP70.

19. A method for increasing expression of a factor having anti-oxidant activity in a cell, the method comprising administering to the cell an effective amount of a compound that inhibits HIF hydroxylase activity, thereby increasing expression of the factor having anti-oxidant activity in the cell.

20. The method of claim 19, wherein the factor having anti-oxidant activity is selected from the group consisting of adrenomedullin, heme oxygenase-1, and HSP70.

21. A method for reducing or preventing oxidative damage in a cell, the method comprising administering to the cell an effective amount of a compound that inhibits HIF hydroxylase activity, thereby reducing or preventing oxidative damage in the cell.

22. A method for conferring cytoprotection to a cell exposed to or at risk for exposure to stress, the method comprising administering to the cell an effective amount of a compound that inhibits HIF hydroxylase activity, thereby conferring cytoprotection to the cell.

23. The method of claim 22, wherein the stress is selected from the group consisting of nutritional imbalance, growth factor imbalance, mechanical stress, thermal stress, reduced oxygen conditions, exposure to free radicals, hypoxia, and ischemia.

24. The method of claim 22, wherein the stress is selected from the group consisting of exposure to a chemical agent, an infectious agent, a toxin, a pollutant, a drug, and radiation.

25. The method of claim 22, wherein the stress is associated with a condition selected from the group consisting of an infection, an inflammation, an immunodeficiency disorder, anaphylaxis, an autoimmune disease, cancer, a neurodegenerative disorder, an aging-associated disorder, heart disease, and cardiac injury.

26. The method of claim 25, wherein the infection is selected from the group consisting of a viral infection and a bacterial infection.

27. The method of claim 22, wherein the stress is associated with a medical procedure or treatment.

28. The method of claim 27, wherein the medical procedure or treatment is selected from the group consisting of radiation therapy, chemotherapy, and surgery.

29. The method according to any of the preceding claims, wherein the compound is selected from the group consisting of a phenanthroline; a heterocyclic carbonyl glycine; a quinoline-2-carboxamide; an isoquinoline-3-carboxamide; and an N-substituted arylsulfonylamino hydroxamic acid.

30. The method according to any of the preceding claims, wherein the compound is selected from the group consisting of 4-Oxo-1,4-dihydro-[1,10]phenanthroline-3-carboxylic acid (Compound A), 3-{[4-(3,3-Dibenzyl-ureido)-benzenesulfonyl]-[2-(4-methoxy-phenyl)-ethyl]-amino}-N-hydroxy-propionamide (Compound B), [(4-Hydroxy-7-phenylsulfanyl-isoquinoline-3-carbonyl)-amino]-acetic acid (Compound C), [(4-Hydroxy-6-phenylsulfanyl-isoquinoline-3-carbonyl)-amino]-acetic acid (Compound D), [(1-Chloro-4-hydroxy-7-phenoxy-isoquinoline-3-carbonyl)-amino]-acetic acid (Compound E), [(1-Bromo-4-hydroxy-7-phenoxy-isoquinoline-3-carbonyl)-amino]-acetic acid (Compound F), [(4-Hydroxy-7-phenoxy-isoquinoline-3-carbonyl)-amino]-acetic acid (Compound G), and [(1-Chloro-4-hydroxy-isoquinoline-3-carbonyl)-amino]-acetic acid (Compound H).
